(19) United States
(12) Patent Application Publication
(10) Pub. No.: US 2019/0111203 A1
(43) Pub. Date: Apr. 18, 2019

(54) DEVICES, SYSTEMS, AND METHODS FOR VESSEL CLEARING

(71) Applicant: ACIST Medical Systems, Inc., Eden Prairie, MN (US)

(72) Inventors: Nicholas S. Mairs, Minneapolis, MN (US); Blaise Damian Porter, Minneapolis, MN (US); Jacob Tyler Williams, St. Louis Park, MN (US)

(21) Appl. No.: 16/158,892

(22) Filed: Oct. 12, 2018

Related U.S. Application Data
(60) Provisional application No. 62/571,871, filed on Oct. 13, 2017.

(51) Int. Cl. A61M 5/00; A61B 8/12; A61B 8/08; A61M 5/168

(52) U.S. Cl. CPC A61M 5/007; A61B 8/12; A61M 2005/1403; A61M 5/16881; A61B 8/481

(57) ABSTRACT

Methods, devices, and systems for clearing a vessel lumen are disclosed. One method of flushing a vessel lumen includes providing a reservoir containing a first fluid at a first pressure. A pressurized second fluid is received at the reservoir from a powered injection system. Receiving the pressurized second fluid at the reservoir pressurizes the first fluid contained in the reservoir to a second pressure that is greater than the first pressure. The first fluid at the second pressure is delivered from the reservoir to the vessel of the patient.

### PRIORITY CLAIM

This application claims priority to U.S. Provisional Patent Application No. 62/571,871 filed Oct. 13, 2017.

### TECHNICAL FIELD

This disclosure generally relates to devices, systems, and methods for clearing a vascular structure.

### BACKGROUND

Vascular diagnostic procedures can be useful in identifying characteristics of a vascular structure, which in turn can be useful in informing decisions related to treatment (e.g., whether an interventional procedure should be performed). One type of vascular diagnostic procedure that can be used to identify diagnostically significant characteristics of a vessel is intravascular imaging. For instance, an intravascular imaging system can be used by a healthcare professional to identify and locate abnormal structures, such lesions, within a vascular structure. Common intravascular imaging systems include intravascular ultrasound (IVUS) systems as well as light-based imaging systems, such as infrared spectroscopy or optical coherence tomography (OCT) systems.

In the example of IVUS, systems can include an ultrasound transducer that emits ultrasound energy. The emitted ultrasound energy can reflect off of one or more vascular structures and be received back at the ultrasound transducer. Upon receiving the reflected ultrasound energy, the ultrasound transducer can generate an electrical signal corresponding to the reflected ultrasound energy. This electrical signal can convey imaging data useful in generating an image of the vessel. In many such systems, a console or other interface component displays generated images in substantially real-time. In this way, IVUS can be used to provide in-vivo visualization of vascular structures and lumens, such as a coronary artery lumen, coronary artery wall morphology, and devices, such as stents, at or near the surface of the coronary artery wall.

### SUMMARY

In many intravascular imaging applications, blood can cause artifacts in image data (e.g., speckle). Therefore, the quality of intravascular imaging data, and thus the quality of the images generated based on this data, can be improved when blood is displaced from the vessel lumen in connection with (e.g., immediately prior to beginning) an intravascular imaging procedure. To displace blood from the vessel lumen, a flushing agent can be introduced into the vessel lumen. In order for the flushing agent to effectively clear blood from the vessel lumen, in many instances a relatively high pressure and/or flow rate is needed when delivering the flushing agent into the vessel lumen. Using a hand manifold (e.g., a hand operated syringe) or a low pressure pump (e.g. a peristaltic pump) may not sufficiently pressurize the flushing agent and/or provide sufficient flow rates for delivering the flushing agent as may be needed in many instances to clear blood from the lumen.

Various embodiments disclosed herein provide devices, systems, and methods for delivering a flushing agent to a vessel lumen to displace, and thereby clear, blood from the vessel lumen. In general, certain embodiments use a powered injection system to provide a first fluid (e.g., a contrast fluid) at a sufficient pressure and/or flow rate along a line in communication with a reservoir containing a second fluid (e.g., a flushing agent, such as a non-contrast fluid). The reservoir receives the first fluid from the powered injection system. Receiving the first fluid at the reservoir pressurizes the second fluid contained in the reservoir, for instance to substantially the pressure of the received first fluid. As a result, such embodiments can use the motive force provided by the powered injector in outputting the first fluid to deliver a second fluid contained in a reservoir downstream from the powered injector at the pressure and/or flow rate provided by the powered injector to the first fluid.

Various embodiments can provide useful advantages in connection with clearing blood from a vessel lumen. For example, certain embodiments can eliminate the need to have a second powered injector to deliver a flushing agent, such as a non-contrast fluid, and thereby reduce costs and user burden barriers to use. Certain embodiments can provide a reservoir containing a flushing agent as an accessory for attachment in-line with a powered injection system, and thereby may provide a low cost user-friendly vessel lumen clearing function. When the reservoir is in the form of an accessory, it can be a modular accessory attached in-line with a variety powered injection system models. Also, some embodiments can include a reservoir containing a flushing agent in the form of a non-contrast fluid, and therefore provide an alternative to contrast fluid vessel clearing, such as in applications where a patient is not suited for receiving contrast fluid in connection with vessel clearing (e.g., a patient having renal insufficiency).

One embodiment includes a method of flushing a vessel of a patient. This particular method embodiment includes providing a reservoir containing a first fluid at a first pressure. This embodiment also includes receiving at the reservoir a pressurized second fluid from a powered injection system. Receiving the pressurized second fluid at the reservoir pressurizes the first fluid contained in the reservoir to a second pressure that is greater than the first pressure. This embodiment further includes delivering the first fluid at the second pressure from the reservoir to the vessel of the patient.

Another embodiment includes a patient tubing system. This embodiment of the patient tubing system includes a first line that has a first end and a second end. The first end is adapted to fluidly connect to a powered injection system to transport a pressurized first fluid from the powered injection system. This embodiment of the patient tubing system also includes a reservoir defining an interior volume with a first portion, a second portion, and a means for sealing the first portion from the second portion. The first portion is in fluid communication with the second end of the fluid line so as to receive the pressurized first fluid. The second portion is adapted to contain a second fluid at a first pressure. Upon receiving the pressurized first fluid the reservoir is adapted to pressurize the second fluid to a second pressure that is greater than the first pressure.

A further embodiment can include an intravascular ultrasound imaging method. This particular method embodiment includes providing an intravascular ultrasound system having a catheter positioned at or proximal to a vessel of a patient, an ultrasound transducer within the catheter, and an imaging engine coupled to the ultrasound transducer. This embodiment also includes providing a powered injection system that pressurizes a first fluid at the powered injection system. The embodiment further includes providing a reservoir that contains a second fluid and is in fluid communication with the powered injection system. Upon receiving the first fluid from the powered injection system, the reservoir increases the pressure of the second fluid contained in the reservoir. The embodiment additionally includes delivering the second fluid at the increased pressure from the reservoir to the vessel of the patient.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a perspective view of an exemplary embodiment of an intravascular ultrasound (IVUS) imaging system.

FIG. 2 is a schematic block diagram of various exemplary components of the IVUS imaging system of FIG. 1.

FIG. 3 is a sectional view of an exemplary embodiment of a catheter assembly for use with the IVUS imaging system of FIG. 1.

FIG. 4 is a perspective view of an exemplary embodiment of a powered injection system.

FIGS. 5A and 5B are schematic block diagrams of exemplary embodiments of a patient tubing system. FIG. 5A shows an embodiment of the patient tubing system without a reservoir, while FIG. 5B shows an embodiment of the patient tubing system with a reservoir in place.

FIG. 6 is a schematic block diagram of another exemplary embodiment of a patient tubing system.

FIG. 7A is a perspective, partially transparent, view of an exemplary embodiment of a reservoir.

FIG. 7B is a side elevational, partially transparent, view of the reservoir of FIG. 7A.

FIG. 8 is a schematic block diagram of a further exemplary embodiment of a patient tubing system having a secondary fluid line.

FIG. 9 is a flow diagram of an exemplary embodiment of a vessel flushing method.

FIG. 10 is a flow diagram of an exemplary embodiment of an intravascular ultrasound imaging method.

### DETAILED DESCRIPTION

The following detailed description is exemplary in nature and provides some practical illustrations and examples. Examples of constructions, materials, dimensions, and manufacturing processes are provided for selected elements, and all other elements employ that which is known to those of ordinary skill in the field. Those skilled in the art will recognize that many of the noted examples have a variety of suitable alternatives.

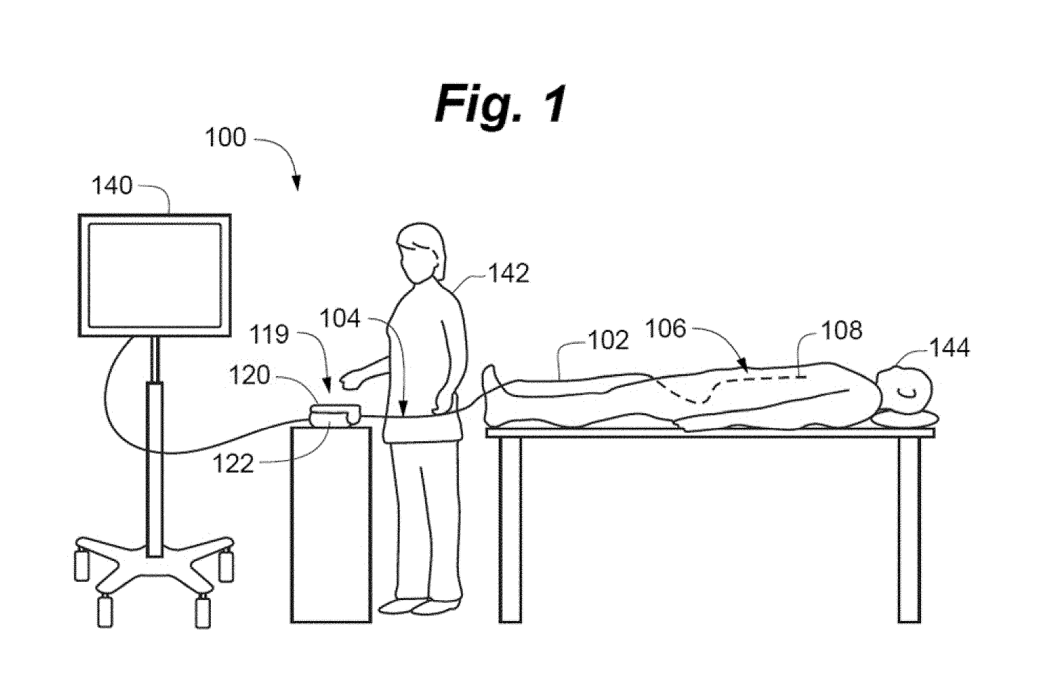

FIG. 1 is an illustrative example of an embodiment of a system 100 that can be configured to perform intravascular imaging. The system 100 can include a catheter assembly 102, a translation device 119, and an imaging engine 140. The catheter assembly 102 can include a proximal end 104 and a distal end 106 configured to be inserted into a vessel of a patient 144. In one example, the catheter assembly 102 can be inserted into the patient 144 via the femoral artery and guided to an area of interest within the patient 144. The broken lines in FIG. 1 represent portions of the catheter assembly 102 within the patient 144.

As shown in FIG. 1, in some examples, the catheter assembly 102 can include an intravascular imaging device 108 at the distal end 106 configured to emit and receive wave-based energy and generate imaging data, such as of an area of interest within the patient 144 to which the distal end 106 is guided. For example, where system 100 is an intravascular ultrasound (IVUS) imaging system, intravascular imaging device 108 can comprise an IVUS imaging probe including an ultrasound transducer that is configured to emit and receive ultrasound energy and thereby convey ultrasound image data. In another example, the system 100 can be an optical coherence tomography (OCT) system wherein the intravascular imaging device 108 can comprise an OCT imaging probe configured to emit and receive light and thereby convey OCT image data.

With continued reference to FIG. 1, the translation device 119 can be configured to translate the intravascular imaging device 108 of the catheter assembly 102. The translation device 119 can comprise, for example, a linear translation system (LTS) 122. As is discussed elsewhere herein, LTS 122 can be mechanically engaged with catheter assembly 102 and configured to translate the catheter assembly 102 a controlled distance within the patient 144 during a translation operation, for instance a pullback or push-forward operation. The system 100 can comprise a patient interface module (PIM) 120 configured to interface the translation device 119 with the catheter assembly 102.

The imaging engine 140 can be in communication with the intravascular imaging device 108 and, in some embodiments, the translation device 119. According to some examples, the imaging engine 140 can comprise at least one programmable processor. In some embodiments, the imaging engine 140 can comprise a computing machine including one or more processors configured to receive commands from a system user 142 and/or display image data acquired from the catheter assembly 102 via a user interface. The computing machine can include computer peripherals (e.g., keyboard, mouse, electronic display) to receive inputs from the system user 142 and output system information and/or signals received from the catheter assembly 102 (e.g., generate an image using image data from the catheter assembly 102). In some examples, the user interface of the computing machine can be a touchscreen display configured to act as both an input device and an output device. In some examples, imaging engine 140 can include memory modules for storing instructions, or software, executable by the processors.

The structure of imagine engine 140 can take a variety of forms. In some embodiments, the imaging engine 140 can be made of an integrated machine that is configured to provide controls for displacing blood from a vessel and subsequently generate blood-displaced vessel images. In certain embodiments, the imaging engine can include separate injection (e.g., for providing high pressure and/or high flow rate fluid) and imaging apparatuses (e.g., for generating blood-displaced images). In some such embodiments involving separate injection and imaging apparatuses, the two separate apparatuses can be configured to communicate and synchronize with one another, for instance to time the injection of fluid as desired with the capture of imaging data within the vessel (e.g., capturing image data within the vessel during or subsequent to clearing blood from the vessel). In some embodiments involving separate injection and imaging apparatuses, the injection apparatus can include a manual injection apparatus.

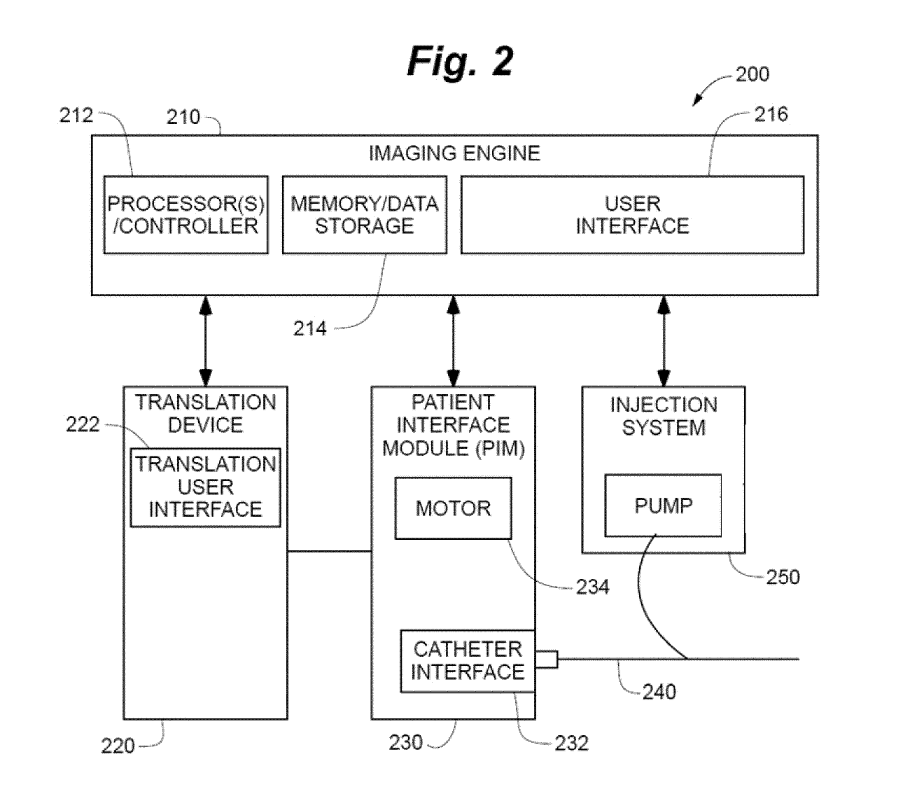

FIG. 2 is a schematic block diagram illustrating various components of a system 200 that can be configured to perform intravascular imaging, such as where the components of the system 200 are part of the system described above in connection with FIG. 1. The system 200 can include an imaging engine 210, a translation device 220, a PIM 230, a catheter assembly 240, and a powered injection system 250. The system 200 can be configured to be used with an OCT, IVUS, and/or other energy-based intravascular imaging device. As discussed further with reference to other figures, in the illustrated embodiment the powered injection system 250 can be in selective fluid commination with the catheter assembly 240. In some such embodiments, the catheter assembly 240 can be of a common catheter configuration, As one example, a common catheter configuration may allow a same catheter structure to be inserted within a vessel for both image data collection (e.g., via the catheter's imaging device) and image data enhancement fluid injection (e.g., via delivery of contrast fluid received from the powered injection system 250 and/or non-contrast fluid received from a device in fluid communication with the powered injection system 250).

According to some examples, the PIM 230 can provide an electromechanical interface between the catheter assembly 240 and the imaging engine 210. In some examples, the PIM 230 can provide a catheter interface 232 to secure the catheter assembly 240 to the system 200. The PIM 230 can include a motor 234 configured to provide mechanical energy to rotate an intravascular imaging device of the catheter assembly 240. According to some examples, the PIM 230 can provide an electrical interface that transmits signals to the intravascular imaging device of the catheter assembly 240 and receives return signals from the intravascular imaging device. In one embodiment, the intravascular imaging device can be electrically rotated, such as via a phased array of ultrasound transducers.

With continued reference to FIG. 2, the translation device 220 can be configured to provide longitudinal translation of the catheter assembly. Translation device 220 can comprise a Linear Translation System (LTS). The translation device 220 can be configured to mate with PIM 230 and catheter assembly 240 to enable controlled pullback of an intravascular imaging device of catheter assembly 240. According to some examples, translation device 220 can feature a translation user interface 222 which can comprise a translation display configured to show translation data associated with the translation of the intravascular imaging device to a user of system 200. In some examples, translation data can include linear distance traversed and/or translation speed. The translation user interface 222 can be configured to receive inputs from a user to control starting/stopping translation, setting translation speed, resetting linear distance traversed to zero, and/or switching to manual mode. In manual mode, a user can freely move the intravascular imaging device of the catheter assembly forward and backward (e.g., distally and proximally). In some examples, the translation device 220 can be configured to enable both pullback and push-forward of the intravascular imaging device at a controlled rate. In another example, the translation device 220 can be configured to oscillate, or cycle, the intravascular imaging device by alternately performing pullback and push-forward operations. In some examples, translation device 220 can include a position sensor configured to measure a distance of a translation operation.

Referring still to FIG. 2, imaging engine 210 of system 200 can comprise one or more processors 212, one or more memory modules 214, and a user interface 216. Imaging engine 210 can be configured to perform one or more functions including, for example, image generation based on image data acquired by the intravascular imaging device, display of intravascular images and other information, control of the system components, storing and exporting the image data, controlling user interface 216 for operating the system 200, analysis tools (e.g., hemodynamic calculations, area measurements, linear measurements, and annotations), and so on. Memory modules 214 can include instructions that can be executed by processors 212 (e.g., software). The memory modules 214 can comprise one or more non-transitory computer readable storage media which can include volatile and/or non-volatile memory forms including, e.g., random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media. In some examples, instructions can be embedded or encoded in the memory modules 214 which can cause processors 212 to perform a method, e.g., when the instructions are executed. In some examples, user interface 216 can be configured to receive inputs from a user of system 200 and can comprise one or more computer peripherals (e.g., keyboard, mouse), voice recognition technology, or other suitable means of receiving inputs from a user. User interface 216 can include a display configured to display imaging data (e.g., intravascular images, system status, hemodynamic measurements) to a user of system 200. In some examples, the user interface 216 can comprise a touch-sensitive screen configured to receive user inputs as well as display imaging data.

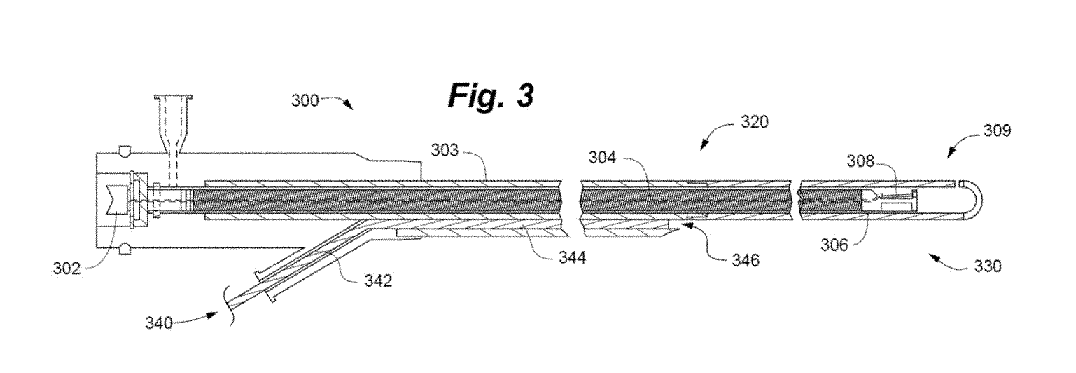

FIG. 3 is a side cross-sectional view of a catheter assembly 300 that can be used, for instance, in system 100 of FIG. 1. A drive cable 304 of the catheter assembly 300 can be mechanically engaged and electrically connected to a patient interface module via a connector 302. Accordingly, the patient interface module can be used to rotate drive cable 304 within sheath 303. Intravascular imaging device 309 can be coupled to drive cable 304 such that rotation of the drive cable 304 causes an imaging element 308 to rotate in a distal section 330 of the catheter assembly 300. The imaging element 308 can be configured to emit and receive wave-based energy and generate imaging data. The imaging data can then be communicated outside of the patient to an imaging engine where the imaging data can be processed to form a generated image. In examples where catheter assembly 300 is configured for use in an IVUS system, imaging element 308 can comprise an ultrasound transducer. In some instances, the ultrasound transducer(s) can operate at a relatively high frequency (e.g., 10 MHz-60 MHz, in some preferred embodiments, 40 MHz-60 MHz) and can be carried near a distal end of an IVUS catheter. Some IVUS systems involve rotating the IVUS catheter (e.g., mechanically, phased-array, etc.) for 360-degree visualization. In examples where catheter assembly 300 is configured for use in an OCT system, imaging element 308 can comprise an OCT imaging probe configured to emit and receive light. In some examples, catheter assembly 300 can include an imaging window 306 substantially transparent to the frequency of the wave-based energy emitted by imaging element 308.

Additionally, the catheter assembly 300 shown in FIG. 3 can include an injection component 340. The injection component 340 can include a catheter fluid inlet to receive fluid 342. The fluid inlet can be in fluid communication, either directly or indirectly, with a powered injection system. In either case, the fluid 342 received at the injection component 340 may be at a relatively high pressure and/or flow rate (e.g., as needed to clear blood from a vessel and/or deliver image enhancing contrast agent(s) to a desired region of a vascular structure). The fluid 342 can be delivered to the vessel via lumen 344 of the injection component 340 to a distal outlet 346. The fluid 342 can be a contrast fluid and/or a flushing agent (e.g., a non-contrast fluid, such as saline). In some applications, the fluid 342 can be delivered via the outlet 346, such as to flush the vessel where the fluid 342 is a flushing agent. Thus, in addition to the illustrated catheter assembly 300 being configured to collect intravascular imaging data, such catheter assembly 300 can also be configured to deliver fluid received from a powered injection system (e.g., indirectly from a component fluidly connected in-line with the powered injection system).

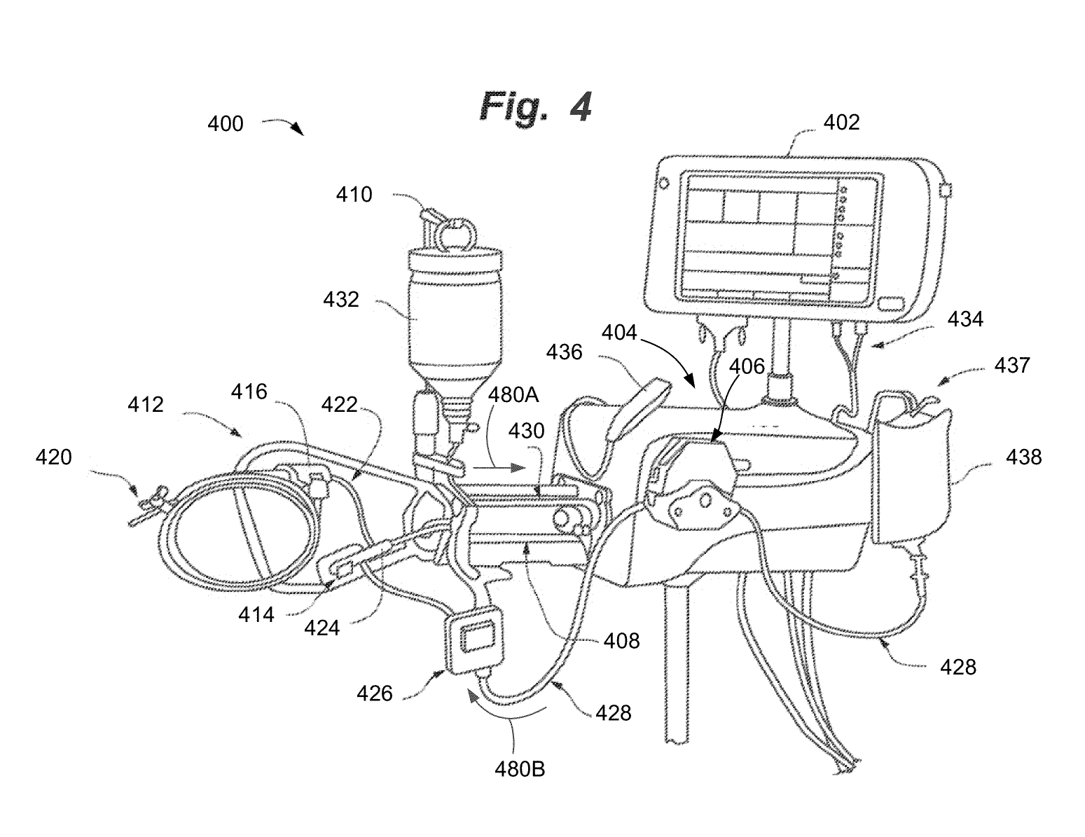

FIG. 4 is a perspective view of an exemplary embodiment of a powered injection system 400. The powered injection system 400 can be used to inject a quantity of fluid into a vessel via a catheter assembly (e.g., the catheter assembly 300 shown and described in connection with FIG. 3). The fluid injected by the system 400 can be a contrast fluid, non-contrast fluid (e.g., saline), or a combination thereof. Exemplary medical procedures performed in connection with the powered injection system 400 can include, OCT imaging, IVUS imaging, angiographic procedures, computed tomography (CT) procedures, magnetic resonance imaging (MRI) procedures, and other forms of diagnostic imaging procedures. The system 400 as illustrated in the example of FIG. 4 includes control panel 402, injector head 404, sleeve 408, reservoir holder 410, module 412, patient manifold sensor 414, and air detector 416.

An operator of system 400 can use control panel 402 to set up various parameters and/or protocols to be used for a given fluid injection procedure. In one example, the operator can interact with control panel 402 to input injection parameters such as flow rate, maximum injection volume, injection pressure (e.g., maximum), rise time, and/or other injection parameters. In one embodiment, control panel 402 includes a touch-screen panel display, enabling an operator to view and modify injection parameters as desired. Control panel 402 can also be used to initialize system 400 (e.g., to prepare it for a patient fluid injection), or to activate certain features or sequences of operations of system 400. The control panel 402 can be controlled by one or more processors, such as processors of the injector head 404. Such processors can also control other components (e.g., injector head 404, a powered injector housed within sleeve 408, pump 406, patient manifold sensor 414, and air detector 416) of system 400.

Holder 410 is capable of holding a fluid container for fluid that can be drawn into the powered injector during operation of system 400. For example, holder 410 can hold a fluid container of contrast fluid. A second holder 437 can hold a flushing agent, container 438 such as a non-contrast fluid. In some cases, the non-contrast fluid can be a diluent (e.g., saline). In some cases, a non-contrast fluid can be delivered via operation of peristaltic pump 406. In the exemplary embodiment illustrated in FIG. 4, tubing from the fluid container is coupled to pump 406. Pump 406 can deliver fluid from the second fluid container through the tubing towards module 412.

Powered injector 430 can pressurize a fluid received within a syringe held thereat, such as the contrast fluid from the contrast fluid reservoir 432 in the illustrated example. In other embodiments, other forms of powered injectors can be used. In the example of FIG. 4, both peristaltic pump 406 and the syringe contained within sleeve 408 are capable of delivering fluid from system 400 to a catheter (e.g., the catheter assembly 300 shown and described in connection with FIG. 3). The syringe contained within sleeve 408 can house a plunger adapted to be driven (e.g., advanced and/or retracted within the syringe) by a motor assembly drive arm which is part of and housed within the injector head 404. As one example, the syringe at sleeve 408 can retract its plunger (e.g., via the motor drive assembly) to draw in contrast fluid from the reservoir 432, and extend its plunger (e.g., via the motor drive assembly) to expel the drawn in contrast fluid along output tubing coupled to an outlet tip of the syringe. The contrast fluid expelled from the syringe may be pressurized by forward motion of the motor-driven plunger, such that the contrast fluid sent along output tubing from the syringe is pressurized. In one application, the contrast fluid can be pressurized by the powered injector 430 anywhere from 1000-1500 psi (e.g., 1200 psi).

In the example shown in FIG. 4, the powered fluid injection system 400 comprises a hand-control device 436 coupled to control panel 402 via a connector 434. In some embodiments, hand-control device 436 can be connected to a component of system 400 other than control panel 402, such as injector head 404. As shown in FIG. 4, hand-control device 436 is coupled to tubing, cabling, or wiring, which connects hand-control device 436 to connector 434, and which allows signals generated by hand-control device 436 to be transmitted or communicated via connector 434. Connector 434 can then be connected to or disconnected from control panel 402, for example, or to some other component of system 400. An operator can manipulate hand-control device 436 to control injection of fluid from system 400. For example, the operator can use hand-control device 436 as a variable-rate control device to variably control the fluid flow rate output from the powered injector 430.

The system 400 may include, in some embodiments, a pressure transducer 426. The pressure transducer 426 can be coupled to tubing 422 and 428 (e.g., high-pressure tubing). The tubing 422 can in turn be connected to a patient line and ultimately a catheter assembly via connector 420. When high-pressure tubing 422 is connected to a patient line, pressure transducer 426 is capable of functioning as a hemodynamic monitor for the patient. Pressure transducer 426 converts detected pressures into electrical signals that can be monitored or otherwise used by system 400.

The system 400 can also include, in some embodiments, an air detector 416. For instance, high-pressure tubing 422 may run through air detector 416. Air detector 416 can be capable of detecting the presence of air (e.g., air bubbles or air columns) within fluid flowing through tubing 422. If air detector 416 detects an undesirable amount of air within the tubing 422, it may generate a signal and send such signal to injector head 404.

Also illustrated in the example of FIG. 4 is a valving system 424. In one example, the valving system 424 can include a manifold valve having a spring-biased spool valve, but in other examples various other types of valves, including check valves, can also be used. An outlet of the valving system 424 can be in communication with the tubing 422. Valving system 424 can be capable of controlling the flow of fluid from powered injector 430 and/or through pump 406 to high-pressure tubing 422, and thus ultimately to the vessel or other device downstream of the powered injection system 400. In one embodiment, for example, when valving system 424 is in a first position fluid can flow from powered injector 430 to tubing 422, but when valving system 424 is in a second position fluid can flow through pump 406 to tubing 422. As such, in some instances, valving system 424 can allow fluid flow to tubing 422 from one of the two sources (e.g., powered injector 430 or peristaltic pump 406) at a time. In this way, where the powered injector 430 provides a relatively high pressure fluid (e.g., contrast fluid at 1000-1500 psi) and the peristaltic pump 405 provides a relatively low pressure fluid (e.g., saline at 25-125 psi), the valving system 424 can selectively convey the high pressure fluid downstream from the system 400.

In various embodiments disclosed herein, a reservoir can be fluidly connected to an output of a powered injection system. The reservoir can be useful, as one example, for delivering a flushing agent to a vessel lumen to displace, and thereby clear, blood from the vessel lumen. For instance, the reservoir can be fluidly connected in-line with, and downstream from, the powered injection system. The reservoir can contain a first fluid (e.g., a flushing agent, such as a non-contrast fluid, for instance saline) at a first pressure. The powered injection system can output a second fluid (e.g., a contrast fluid) at a sufficient pressure (e.g., 1000-1500 psi) and/or flow rate along a line in fluid communication with the reservoir. The reservoir may receive the second fluid from the powered injection system. Upon receiving the second fluid from the powered injection system, the first fluid contained in the reservoir is pressurized to a second pressure that is greater than the first pressure (e.g., to substantially the pressure of the received first fluid). The first fluid at the second pressure can be delivered from the reservoir to the vessel of the patient. Thus, in one example the reservoir can use the motive force provided by the powered injector to deliver fluid contained in the reservoir at the pressure and/or flow rate provided by the powered injector to fluid output from the powered injector.

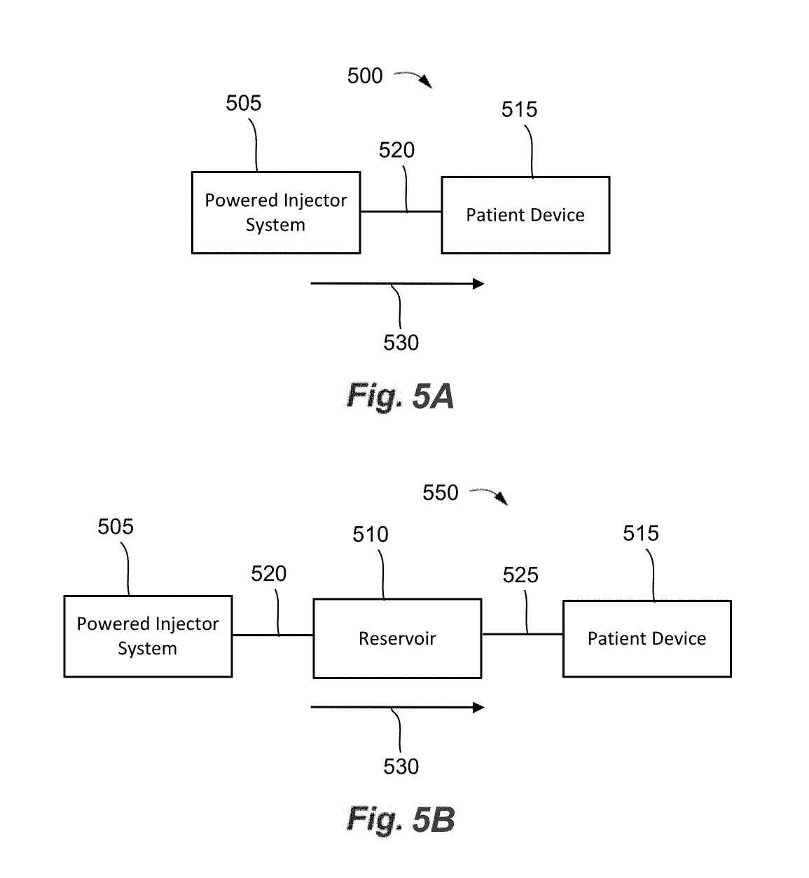

FIGS. 5A and 5B show schematic block diagrams of exemplary embodiments of a patient tubing system. FIG. 5A shows an embodiment of a patient tubing system 500 without a reservoir, while FIG. 5B shows an embodiment of a patient tubing system 550 with a reservoir in place.

The illustrated embodiment of the system 500 includes a powered injection system 505 and a patient device 515. The powered injection system 505 is in fluid communication with the reservoir 510 via a line 520. Flow direction in the system 500 is indicated by the arrow 530. The powered injection system 505 can include any device, or combination of devices, adapted to output fluid at a pressure and/or flow rate sufficient to flush (e.g., clear) blood from a vessel of a patient. As one example, the powered injection system 505 can be the same as, or similar to, powered injection systems described previously herein. The patient device 515 can include any device, or combination of devices, adapted to receive fluid and deliver such fluid to an anatomical structure of a patient (e.g., to an internal anatomical structure of a patient, such as a vessel lumen). As one example, the patient device 515 can be a catheter assembly, such as that described previously herein (e.g., for use with an IVUS imaging system).

As described previously, fluid is pressurized by the powered injector system 505. This pressurized fluid is output from the powered injector system 505 along the line 520 and delivered to the patient device 515. The patient device 515 is adapted to deliver this pressurized fluid to a patient (e.g., to a vessel of the patient).

The illustrated embodiment of the system 550 in FIG. 5B shows how a reservoir 510 can be easily positioned within the system described for FIG. 5A. As shown, the reservoir 510 is removably connected between the powered injector system 505 and the patient device 515. An additional line 525 may also be used in some cases when adding the reservoir 510 to the system 550.

In the embodiment of the system 550 of FIG. 5B, a first fluid at a first pressure (e.g., atmospheric pressure, greater than atmospheric pressure) is contained within the reservoir 510. In one application, the first fluid contained in the reservoir 510 can be a non-contrast fluid (e.g., comprising saline). A second fluid is pressurized by the powered injector system 505 and output from the powered injector system 505 along the line 520. In one application, the second fluid can be different from the first fluid, for example where the first fluid is a non-contrast fluid the second fluid can be a contrast fluid. The pressurized second fluid is received at the reservoir 510 via the line 520. Receiving the second fluid at the reservoir 510 pressurizes the first fluid contained in the reservoir 510 to a second pressure that is greater than the first pressure. The first fluid at the second pressure is output from the reservoir on line 525 and delivered to the patient device 515. The patient device 515 is adapted to deliver the first fluid at the second pressure to a patient (e.g., to a vessel of the patient).

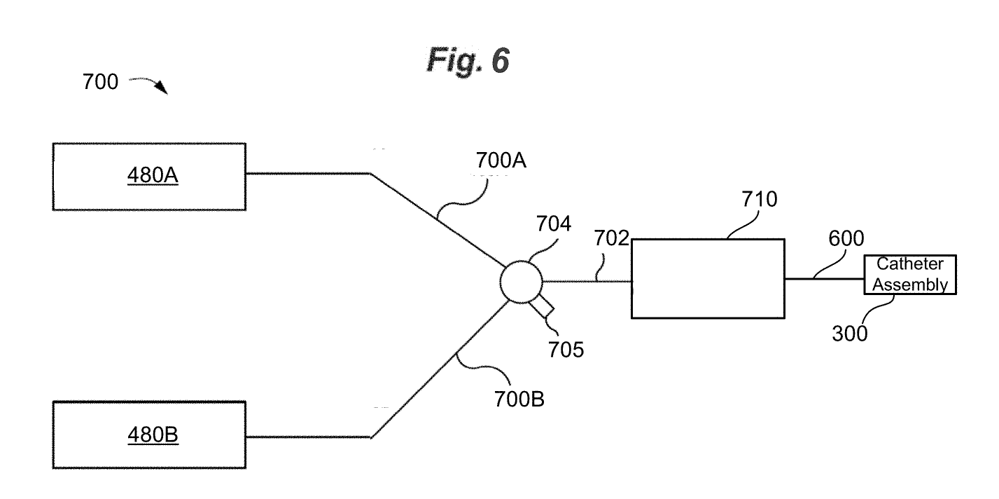

FIG. 6 shows a schematic block diagram of another exemplary embodiment of a patient tubing system 700 that similarly includes an exemplary reservoir 710. In the illustrated example of FIG. 6, the patient tubing system 700 can deliver one or more fluids from the powered injector system to the reservoir 710, which can then convey a same or different fluid on to a catheter assembly, such as the catheter assembly 300. As shown, the patient tubing system 700 has a contrast fluid line 700A and non-contrast fluid line 700B. In this example, the fluid line 700A and 700B form a first line that fluidly connects the powered injector system on a first end to the reservoir 710 on a second end. In other example, this first line can be a single tubing line extending from just one of the contrast fluid source and the non-contrast fluid source (e.g., a first line extending from only the powered injector) to the reservoir 710. The contrast fluid line 700A can be fluidly coupled to and positioned downstream of the contrast fluid source 480A (e.g., the powered injector having the pressurized syringe and associated contrast fluid container), and thus the powered injection system. The non-contrast fluid line 700B can be fluidly coupled to and positioned downstream of the non-contrast fluid source 480B. As one example, the non-contrast fluid source 480B can include a fluid path originating from the non-contrast fluid reservoir, associated tubing, and peristaltic pump. The non-contrast fluid line 700B can thus be positioned in some cases downstream of the non-contrast fluid source 480B.

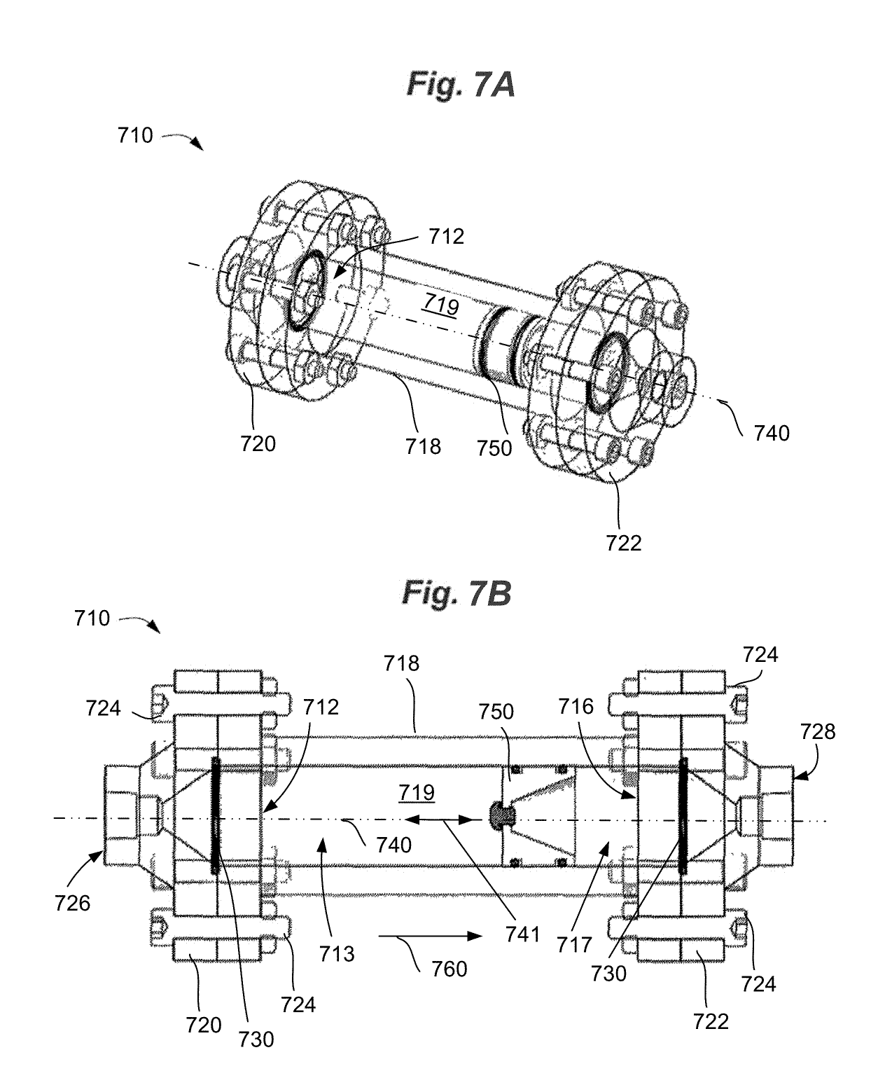

As also shown in the example of FIG. 6, the patient tubing system 700 further has a valving system 704 adapted to selectively fluidly couple an outlet line 702 and the contrast fluid line 700A or the non-contrast fluid line 700B. As such, the reservoir 710 in the illustrated example can receive contrast fluid from the fluid line 700A and/or the non-contrast fluid from the fluid line 700B. The outlet line 702 and valving system 704 may form a portion of the first line connecting the powered injector and the reservoir 710. The valving system 704 shown in the embodiment of FIG. 7 is positioned downstream of the contrast fluid line 700A and the non-contrast fluid line 700B. The valving system 704 can take a variety of forms, and as one example can be a spring-based manifold valve. Such spring-based manifold valve may have a valve member biased so as to fluidly couple the non-contrast fluid line 700B to the outlet line 702 and switch to couple the contrast fluid line 700A to the outline line 702 when a fluid pressure generated along the contrast fluid line 700A becomes great enough to overcome the bias force (e.g., when the powered injector has the plunger driven forward by the motor drive to expel pressurized contrast fluid).

In addition to the reservoir 710 being fluidly coupled to the first line extending from the powered injection system, the reservoir 710 can also be fluidly coupled to a second line, such as a patient line 600. The patient line 600 can extend from the reservoir 710 to the catheter assembly 300, and thereby deliver fluid from the reservoir 710 to the vessel via the catheter assembly 300.

As noted, the reservoir 710 can be positioned in-line and in fluid communication with the powered injection system and can thereby receive a first fluid from the powered injection system (e.g., a contrast fluid from the motor-driven syringe of powered injector). The reservoir 710 can contain a second fluid (e.g., a flushing agent, such as a non-contrast fluid) within an interior volume thereof. The reservoir 710 can initially have the second fluid contained therein, for instance via non-contrast fluid line 700B or via an external fill operation. The reservoir 710 can receive the first fluid at a pressure (e.g., greater than 1000 psi, between 1000 and 1500 psi) and/or flow rate imparted to the fluid by the powered injection system. Receiving the pressurized first fluid at the reservoir 710 pressurizes the second fluid contained in the reservoir 710. Then, the pressurized second fluid can be output from the reservoir 710 on the patient line 600 and ultimately delivered to the vessel lumen at substantially the pressure and/or flow rate of the received first fluid. The reservoir 710 may receive the first fluid (e.g., contrast fluid) from the powered injection system and use it to pressurize the second fluid (e.g., non-contrast fluid) contained therein.

FIGS. 7A and 7B show perspective and side elevational views, respectively, of one example configuration of the reservoir 710. FIGS. 7A and 7B are illustrated as partially transparent in order to show certain internal features of the reservoir 710. The reservoir 710 can define an inlet port 712 fluidly coupled to the first line, such an outlet line portion thereof in some cases. In such instances, the inlet port 712 can be configured to receive pressurized fluid from the powered injection system. The reservoir 710 can also define an outlet port 716 that can be fluidly coupled to a second line, such as a patient line portion thereof in some cases. The reservoir 710 can include walls 718. Outer walls 718 can be made of a polymer such as polycarbonate. The outer walls 718 may define an interior volume 719. The interior volume 719 can be in fluid communication with one or both of the inlet port 712 and the outlet port 716. For instance, the interior volume may include a first portion 713 in fluid communication with the inlet port 712 and a second portion 717 in fluid communication with the outlet port 716.

The illustrated example of the reservoir 710 further has an inlet cap 720 on an inlet side 726 and outlet cap 722 on an outlet side 728. Each of the inlet cap 720 and the outlet cap 722 can be connected to the reservoir 710 via a suitable coupling mechanism, for instance bolts 724 as shown in the example of FIGS. 7A and 7B. The cap 720 and/or 722 can include a seal 730 (e.g., O-rings, or other fluid impermeable sealing materials) placed proximate to the respective inlet and/or outlet ports 712, 716 of the reservoir 710 to prevent leakage of fluid contained within the interior volume 719. Seals 730 can be useful in embodiments where the reservoir 710 is prefilled with fluid, for instance the non-contrast fluid. When included, the caps 720, 722 can be removed when connecting the reservoir 710 in-line with the powered injector system so as to not impede fluid communication between the reservoir 710 and the powered injection system at the inlet port 712 and the reservoir 710 and the vessel at the outlet port 716.

As also shown in the exemplary embodiment illustrated in FIGS. 7A and 7B, the reservoir 710 can include a movable barrier 750. Arrow 741 is included in FIG. 7B to illustrate directional movement of the movable barrier 750. As shown, the movable barrier 750 can be located within the interior volume 719 in some embodiments. When so included, the movable barrier 750 can, in some designs, seal the first portion 713 of the interior volume 719 at the inlet port side 712 from the second portion 717 of the interior volume 719 at the outlet port side 716. In this way, the movable barrier can prevent fluid contact between the pressurized first fluid received at the inlet port 712 and the second fluid contained within the second portion 717 of the interior volume 719. The movable barrier 750 can translate along the interior volume 719 of the reservoir 710 and thereby act to dynamically define the size of the first and second portions 713, 717 within the interior volume 719 on each side of the movable barrier 750. For instance, the movable barrier 750 can move within the inner volume 719 along a longitudinal axis 740 of the reservoir 710 and while so moving can continually alter the volumes of the first and second portions 713, 717 within the interior volume 719.

In embodiments of the reservoir 710 including the movable barrier 750, the movable barrier 750 can be adapted to pressurize the second fluid (e.g. a flushing agent such as non-contrast fluid) upon the reservoir 710 receiving the first fluid pressurized by the powered injector system. As noted, the second fluid can be contained in the second portion 717 of the interior volume 719 defined on the outlet port 716 side of the movable barrier 750. When the reservoir 710 is connected in-line with the powered injection system at the inlet port 712, the first fluid pressurized by the powered injection system can be received into the first portion 713 of the interior volume 719 defined on the inlet side of the movable barrier 750. Receiving the first pressurized fluid at the reservoir 710 can cause the movable barrier 750 to come into contact with this first pressurized fluid and move along the axis 740 in the direction 760 (e.g., in a direction toward the outlet port 716) as shown in FIG. 7B. This movement of the movable barrier 750 upon contact with the first pressurized fluid can cause the second portion 717 of the interior volume 719 to decrease in volume. As a result, the movement of the movable barrier 750 upon contacting the pressurized first fluid can pressurize the second fluid. In various embodiments, the reservoir 710 can pressurize the second fluid contained therein upon the reservoir 710 receiving the pressurized first fluid from the powered injector system to a substantially similar pressure as that of the received first fluid (e.g., that pressure imparted to the first fluid by the powered injector).

In other embodiments, the reservoir 710 need not seal the first and second portions 713, 717 of the interior volume 719, and thus can serve to mix first and second fluids in some cases. In one example, a check valve can be included within the interior volume 719 for such purpose. In such embodiments the second fluid that is pressurized and delivered from the reservoir 710 can be a diluted concentration of the first fluid that is received at the reservoir 710 from the powered injector system.

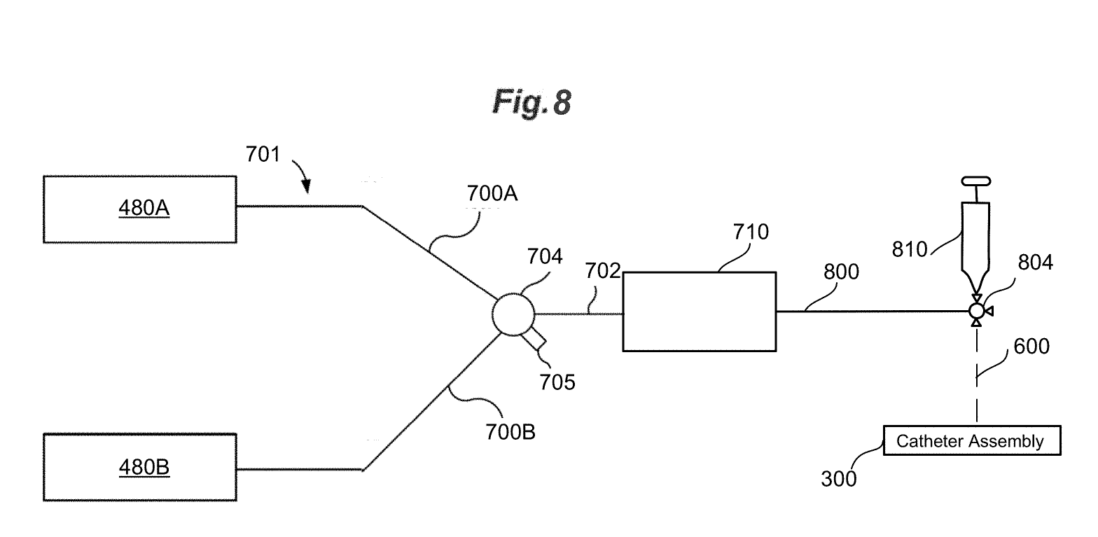

FIG. 8 shows a schematic block diagram of a further embodiment of a patient tubing system, similar to that shown and described previously, but additionally including a secondary fluid line 800. The secondary fluid line 800 can be coupled between the reservoir 710 (e.g., the outlet port of the reservoir 710) and the patient line 600. As such, the secondary fluid line 800 can be in fluid communication with the outlet port of the reservoir 710, such as to fill the reservoir 710 with a bolus of the second fluid. Alternatively or additionally, the secondary fluid line 800 can be used for purging fluids from the reservoir 710, or delivering additional fluid(s) to the catheter assembly 300 via the patient line 600. In some embodiments, a downstream valve system 804 can be provided in fluid communication with the secondary fluid line 800 and with the patient line 600 so as to selectively fluidly couple the secondary fluid line 800 or a syringe 810 to the patient line 600. The downstream valve system 804 can be controlled such that an operator can perform at least one of (i) purging the pressurized second fluid out of the reservoir 710; and (ii) refilling the reservoir 710 with a bolus of substantially non-pressurized second fluid that is to subsequently be pressurized by the reservoir 710 as described previously. The downstream valve system 800 may also facilitate simultaneously filling of the reservoir 710 with pressurized first fluid from the outlet line 702 while the second fluid is delivered to the vessel.

As noted previously, the catheter assembly 300 can receive the second fluid, pressurized at the reservoir 710, via the patient line 600 and deliver the pressurized second fluid to a vessel (not shown) of the patient. The second fluid as pressurized at the reservoir 710 and delivered to the vessel can perform a vessel flushing operation (e.g., sometimes referred to as “vessel clearing”) to displace blood from a vessel lumen. When blood has been cleared as desired, the reservoir 710 and/or associated fluid lines, valve assemblies, and catheter assemblies can be disconnected and may be discarded. As one example, the reservoir can be disconnected from the outlet line after delivering the first fluid at the second pressure to the patient, and the outlet line can then be connected to the contrast fluid line.

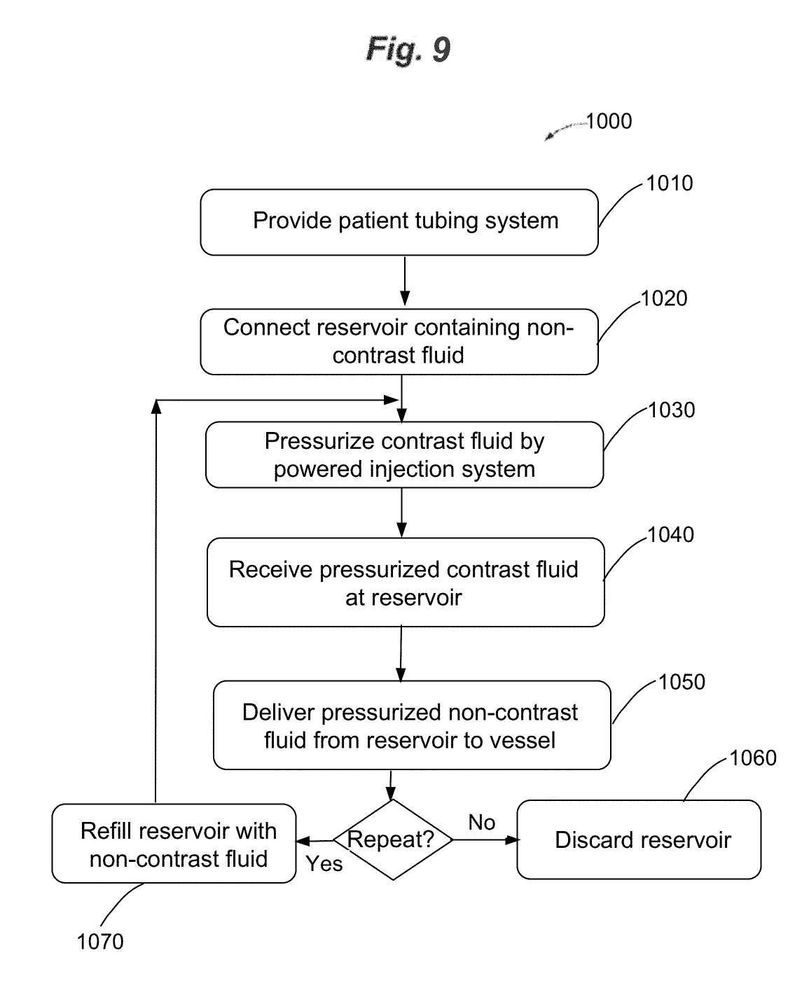

FIG. 9 shows a flow chart of an exemplary embodiment of a vessel flushing method 1000, such as with a flushing agent (e.g., non-contrast fluid such as saline). As seen in FIG. 9, the method 1000 includes providing a patient tubing system at step 1010. The provided patient tubing system can include, for instance, any such tubing system disclosed herein. The method can further include at step 1020 connecting a reservoir containing a non-contrast fluid. This reservoir can, for example, be connected to a powered injector system, for instance via a first tubing line (e.g., an outlet line, valving assembly, contrast line, and/or non-contrast line, and any combinations thereof). At step 1030, the method can also include pressurizing a contrast fluid at the powered injector system, such as described herein. At step 1040, the method may additionally include receiving the pressurized contrast fluid from the powered injector system at the reservoir. This step can include using the received pressurized contrast fluid at the reservoir to pressurize the non-contrast fluid contained in the reservoir (e.g., to substantially the same pressure as the received contrast fluid). At step 1050, the method can further include delivering the pressurized non-contrast fluid from the reservoir to a vessel. In some cases, the pressurized non-contrast fluid can reach the vessel and serve to clear blood contents from within a vessel lumen. This may facilitate a subsequent intravascular imaging procedure.

In some embodiments, the method 1000 can additionally include a step 1070 that involves refilling the reservoir with non-contrast fluid (e.g., non-contrast fluid at a first pressure that is less than a second pressure to which the non-contrast fluid will be pressurized within the reservoir), and repeating steps 1030 on as desired. In one example, the reservoir can be refilled with non-contrast fluid while delivering pressurized non-contrast fluid from the reservoir. As one optional step, the method 1000 can include at step 1060 discarding the reservoir.

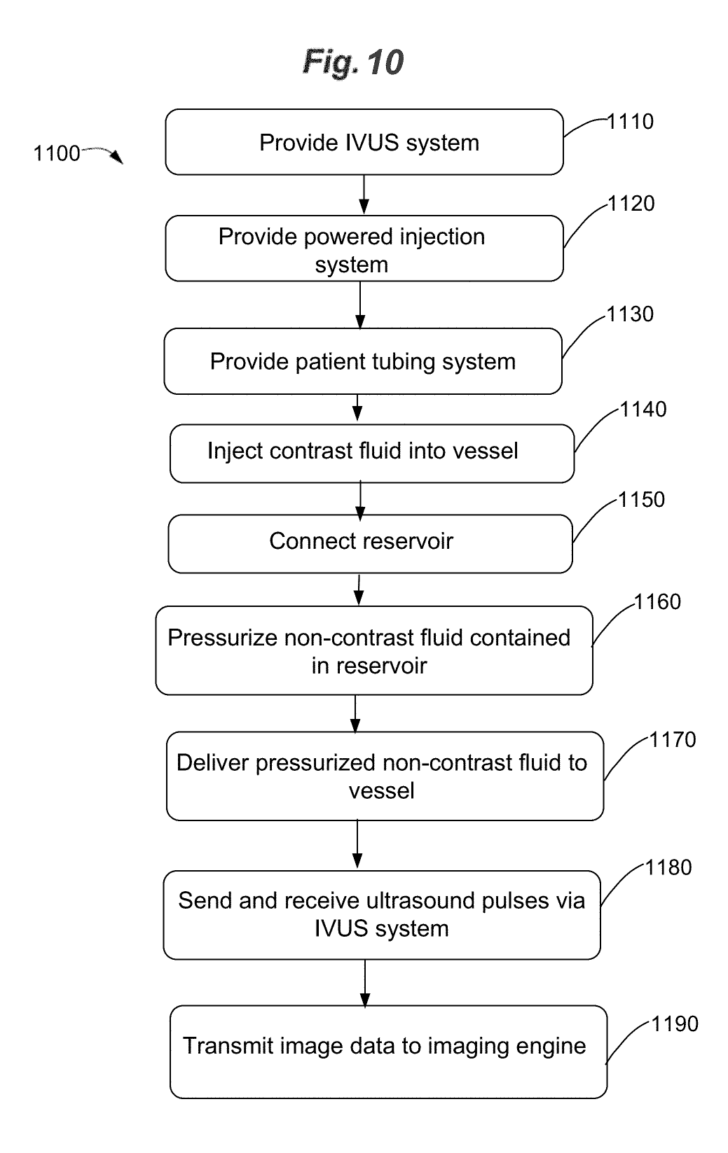

FIG. 10 shows a flow diagram of an exemplary embodiment of a method 1100 for intravascular ultrasound imaging. The method can include at step 1110 providing an intravascular ultrasound (IVUS) system. A provided IVUS system can include, for example, any of those such systems disclosed herein. The method can further include at step 1120 providing a powered injection system. A provided powered injection system can include, for example, any of those such systems disclosed herein. In some examples, the IVUS system and the powered injection system can be provided by the same overall piece of equipment, and thus steps 1110 and 1120 may be the same (i.e. a single step). The method can also include at step 1130 providing a patient tubing system. A provided patient tubing system can include, for example, any of those such systems disclosed herein. The patient tubing system can be provided, at least in part, in communication with the powered injector system. The method may additionally include at step 1140 injecting a contrast fluid from the powered injector system into a vessel lumen. The contrast fluid can be injected into the vessel, for instance, to facilitate placement of an IVUS imaging catheter within the vessel lumen. In some embodiments of the method 1100, step 1140 may not be performed. The method can further include at step 1150 connecting a reservoir containing non-contrast. This reservoir can, for example, be connected to a powered injector system, for instance via a first tubing line (e.g., an outlet line, valving assembly, contrast line, and/or non-contrast line, and any combinations thereof). At step 1160, the method can include pressurizing a non-contrast fluid contained in the reservoir. The reservoir can be in fluid communication with the powered injection system, and the non-contrast fluid contained in the reservoir can be pressurized at the reservoir upon receiving contrast fluid from the powered injection system at the reservoir. The method can include at step 1170 delivering the pressurized non-contrast fluid from the reservoir to the vessel lumen. Receiving the pressurized non-contrast fluid at the vessel lumen can act to flush the vessel lumen of blood contents. The method can additionally include at step 1180 sending and receiving ultrasound pulses at the IVUS system. For instance, ultrasound energy can be emitted, reflected off a vascular lumen structure, and received back at an ultrasound transducer of a catheter assembly to acquire image data pertaining to the vessel.

Certain embodiments of the method 1100 can further include step 1190. At step 1190, image data acquired within the vessel lumen is transferred to an imaging engine. The imaging engine may process this imaging data and display an intravascular image.

In one specific application, embodiments of the patient tubing systems and methods described herein can facilitate increasing the pressure of a non-contrast fluid contained at a location separate from the powered injector system by using the pressure provided by the powered injector system. For instance, the non-contrast fluid contained at the location separate from the powered injector system can be pressurized equal to or greater than 1000 psi, such as between 1000 psi and 1500 psi when fluid from the powered injector system is received at the location of the non-contrast fluid. This can provide adequate pressures and flow rates for performing vessel flushing, for example, prior to IVUS imaging, without needing to employ multiple powered injector systems.

Various examples have been described in considerable detail with reference to certain disclosed embodiments. The embodiments are presented for purposes of illustration and not limitation. One skilled in the art will appreciate that various changes, adaptations, and modifications can be made without departing from the scope of the appended claims.

1. A method of flushing a vessel of a patient, the method comprising the steps of:
providing a reservoir containing a first fluid at a first pressure;
receiving at the reservoir a pressurized second fluid from a powered injection system, wherein receiving the pressurized second fluid at the reservoir pressurizes the first fluid contained in the reservoir to a second pressure that is greater than the first pressure; and
delivering the first fluid at the second pressure from the reservoir to the vessel of the patient.

2. The method of claim 1, wherein the first fluid and the pressurized second fluid are different fluids, the first fluid being a non-contrast fluid and the pressurized second fluid being a contrast fluid.

3. The method of claim 1, further comprising the steps of:
providing a patient tubing system, the patient tubing system comprising:
a contrast fluid line coupled to the powered injection system,
a non-contrast fluid line, and
a valving system fluidly coupled to the non-contrast fluid line, the contrast fluid line, and an outlet line; and
connecting the reservoir to the outlet line, the reservoir having a patient line that extends from the reservoir to the vessel of the patient,
wherein the pressurized second fluid is conveyed from the powered injection system through the contrast fluid line, across the valving system, and through the outlet line to the reservoir, and wherein the first fluid at the second pressure is conveyed from the reservoir through the patient line to the vessel of the patient.

4. The method of claim 3, further comprising the step of:
disconnecting the reservoir from the outlet line after delivering the first fluid at the second pressure from the reservoir to the vessel of the patient; and
connecting the outlet line to the vessel of the patient.

5. The method of claim 1, further comprising the steps of:
after delivering the first fluid at the second pressure from the reservoir to the vessel of the patient, refilling the reservoir with additional first fluid at the first pressure;
receiving at the reservoir the pressurized second fluid from the powered injection system, wherein receiving the pressurized second fluid at the reservoir pressurizes the additional first fluid contained in the reservoir to a third pressure that is greater than the first pressure; and
delivering the additional first fluid at the third pressure from the reservoir to the vessel of the patient.

6. The method of claim 1, further comprising the step of:
performing intravascular ultrasound imaging in the vessel of the patient after delivering the first fluid at the second pressure from the reservoir to the vessel of the patient.

7. The method of claim 1, wherein the first fluid contained in the reservoir is pressurized to the second pressure by contacting a movable barrier within the reservoir with the pressurized second fluid such that the movable barrier acts on the first fluid when contacted by the pressurized second fluid.

8. The method of claim 7, wherein the movable barrier provides a fluid seal between a first portion of the reservoir containing the first fluid and a second portion of the reservoir receiving the pressurized second fluid.

9. The method of claim 1, wherein the second pressure of the first fluid is between 1000 psi and 1500 psi.

10. A patient tubing system comprising:
a first line that includes a first end and a second end, the first end being adapted to fluidly connect to a powered injection system to transport a pressurized first fluid from the powered injection system; and
a reservoir defining an interior volume with a first portion, a second portion, and a means for sealing the first portion from the second portion, the first portion being in fluid communication with the second end of the fluid line so as to receive the pressurized first fluid, the second portion being adapted to contain a second fluid at a first pressure, wherein upon receiving the pressurized first fluid the reservoir is adapted to pressurize the second fluid to a second pressure that is greater than the first pressure.

11. The patient tubing system of claim 10, wherein the means for sealing the first portion from the second portion comprises a movable barrier.

12. The patient tubing system of claim 11, wherein the movable barrier of the reservoir is adapted to pressurize the second fluid to the second pressure upon the first portion of the interior volume receiving the pressurized first fluid.

13. The patient tubing system of claim 12, wherein the movable barrier is adapted to pressurize the second fluid to the second pressure upon the first portion of the interior volume receiving the pressurized first fluid by moving within the interior volume of the reservoir when contacted by the pressurized first fluid.

14. The patient tubing system of claim 13, wherein the movable barrier is adapted to begin moving within the interior volume of the reservoir when contacted by the pressurized first fluid at a pressure of 1000 psi or greater.

15. The patient tubing system of claim 13, wherein a volume of the second portion of the interior volume decreases as the movable barrier is moved within the interior volume of the reservoir upon contact with the pressurized first fluid.

16. The patient tubing system of claim 10, further comprising a patient delivery line that includes a first end and a second end, the first end being in fluid communication with the second portion of the interior volume of the reservoir, the second end being in fluid communication with a catheter.

17. The patient tubing system of claim 16, further comprising means in fluid communication with the patient delivery line for performing at least one of: (i) purging the second fluid at the second pressure out of the second portion of the interior volume of the reservoir, and (ii) refilling the second portion of the interior volume of the reservoir with a quantity of the second fluid at the first pressure.

18. The patient tubing system of claim 17, wherein the means in fluid communication with the patient delivery line for performing at least one of the purging and refilling comprises a downstream valve system fluidly connected to a syringe.

19. The patient tubing system of claim 16, wherein the patient delivery line is adapted to deliver the second fluid at the second pressure to a vessel of a patient through the catheter.

20. The patient tubing system of claim 10, wherein the first line comprises:
a contrast fluid line including the first end of the first line and adapted to couple to the powered injection system,
a non-contrast fluid line; and
a valving system coupled to the contrast fluid line, the non-contrast fluid line, and an outlet line, wherein the valving system is adapted to selectively couple the contrast fluid line to the outlet line or the non-contrast fluid line to the outlet line, and wherein the outlet line includes the second end of the first line and is adapted to fluidly connect to the reservoir.
